(19) United States
(12) Patent Application Publication
(10) Pub. No.: US 2025/0372249 A1
(43) Pub. Date: Dec. 4, 2025

(54) Computer-Implemented Method and System for Telemedicine and Information Retrieval

(71) Applicant: Freddie Figgers, Fort Lauderdale, FL (US)

(72) Inventor: Freddie Figgers, Fort Lauderdale, FL (US)

(21) Appl. No.: 18/731,310

(22) Filed: Jun. 2, 2024

(51) Int. Cl. G16H 40/67; G06F 21/31; G06F 21/62; G06F 40/20; G16H 10/20; G16H 10/60; G16H 50/20

(52) U.S. Cl. CPC G16H 40/67; G06F 21/31; G06F 21/6245; G06F 40/20; G16H 10/20; G16H 10/60; G16H 50/20

(57) ABSTRACT

A computer-implemented method for facilitating telemedicine service is provided. The method includes receiving, by a server computer from a client device over a network, a user request for telemedicine services, wherein the user request is received through a chatbot application for facilitating the telemedicine service. The method includes analyzing the user request using an AI application on the server computer, wherein the AI application includes machine learning algorithms configured to interpret the user request. The method includes generating a response to the user request based on the analysis. The method includes transmitting the generated response to the client device through the chatbot application.

### FIELD OF THE INVENTION

The present invention relates generally to the field of telemedicine, and more specifically to a computer-implemented method and system for telemedicine and information retrieval.

### BACKGROUND

Telemedicine has transformed the healthcare industry by enabling remote access to medical services, consultations, and information. Telemedicine offers a valuable alternative to traditional in-person healthcare visits. This transformation has been significant in recent years, driven by advancements in technology, increased Internet connectivity, and the need for more accessible and efficient healthcare solutions.

Despite the numerous advantages of telemedicine, several drawbacks exist in currently existing systems. Many existing telemedicine systems offer generic solutions for a wide range of healthcare needs. These systems often fail to provide personalized healthcare guidance tailored to the individual patient's specific health issues and circumstances. The absence of a personalized approach can lead to suboptimal user experiences and healthcare outcomes.

Current telemedicine systems do not incorporate artificial intelligence and machine learning to analyze patient requests and provide appropriate responses. This limits the system's ability to offer timely, accurate, and proactive healthcare guidance, often resulting in delayed or inadequate medical advice. Also, without AI, these systems cannot efficiently analyze medical data to assist in retrieval of most relevant data responsive to patient requests.

Also, current telemedicine systems may not provide comprehensive support for users in selecting the most suitable healthcare site. Users may struggle to identify the best healthcare facility based on their unique needs, such as medical specialization, location, and services offered. Also, current telemedicine systems may not offer detailed navigation guidance, causing frustration for patients trying to reach their chosen healthcare site.

### SUMMARY

An illustrative embodiment provides a computer-implemented method for facilitating telemedicine service. The method includes receiving, by a server computer from a client device over a network, a user request for telemedicine services, wherein the user request is received through a chatbot application for facilitating the telemedicine service. The method includes analyzing the user request using an AI application on the server computer, wherein the AI application includes machine learning algorithms configured to interpret the user request. The method includes generating a response to the user request based on the analysis. The method includes transmitting the generated response to the client device through the chatbot application.

In an illustrative embodiment, the method includes receiving a request from the client device to access electronic health records. The method includes authenticating the user using a user validation application configured to verify the user's identity. The method includes providing access to the user to the electronic health records upon authentication.

In an illustrative embodiment, a computer-implemented method and system for facilitating healthcare site selection and information retrieval is provided. The method comprises receiving, from a user, one or more user queries related to healthcare needs through a chatbot application. The method comprises presenting predefined questions to the user for selection based on the user's current health issue or reason for seeking assistance. The method comprises providing a response based on the selected predefined question and offering a list of approved healthcare sites to the user. The method comprises receiving the user's selection of a healthcare site from the list and presenting additional information about the selected healthcare site, including directions to the site.

In an illustrative embodiment, the user's selection determines the subsequent information provided by the chatbot application.

In an illustrative embodiment, the chatbot application processes the user's queries to extract information including the type of healthcare provider required and location-related criteria to refine the search for suitable healthcare sites.

In an illustrative embodiment, the additional information provided about the selected healthcare site includes address, contact information, operating hours, and specific user preparations.

In an illustrative embodiment, a system for facilitating healthcare site selection and information retrieval comprises a chatbot application configured to receive user queries related to healthcare needs and provide predefined questions for user selection based on the user's health issue and reason for seeking assistance. The system comprises a storage unit comprising a database of healthcare providers for searching and identifying suitable healthcare sites based on user preferences and location. The system comprises means for presenting a list of approved healthcare sites to the user and receiving the user's selection. The system comprises means for providing additional information about the selected healthcare site, including directions to the site.

In an illustrative embodiment, the storage unit comprises a match database configured to connect the user with one or more suitable healthcare sites.

In an illustrative embodiment, the system comprises a secure messaging application configured to facilitate exchange of messages between the user and the chatbot application.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 depicts a pictorial representation of a network of a data processing system in which illustrative embodiments may be implemented;

FIG. 2 provides a block diagram of a system in accordance with an illustrative embodiment;

FIG. 3 illustrates a storage unit in accordance with an illustrative embodiment;

FIGS. 4A, 4B, 5A and 5B illustrate flowcharts of various processes in accordance with an illustrative embodiment;

FIGS. 6-12 show graphical user interfaces of a chatbot application in accordance with an illustrative embodiment;

FIG. 13 illustrates a flowchart for processing user queries in accordance with an illustrative embodiment;

FIG. 14 is a block diagram for exchanging messages between a user and a chatbot application in accordance with an illustrative embodiment;

FIGS. 15A-15B illustrate graphical user interfaces through which a user may send messages as a named individual and anonymously;

FIG. 16 illustrates a sequence of messages exchanged between a user, backend services and email services in accordance with an illustrative embodiment; and

FIG. 17 illustrates a block diagram of a data processing system in accordance with an illustrative embodiment.

### DETAILED DESCRIPTION

The illustrative embodiments provide a computer-implemented method and system for facilitating telemedicine service and information retrieval. The illustrative embodiments address the limitations associated with current methods and systems.

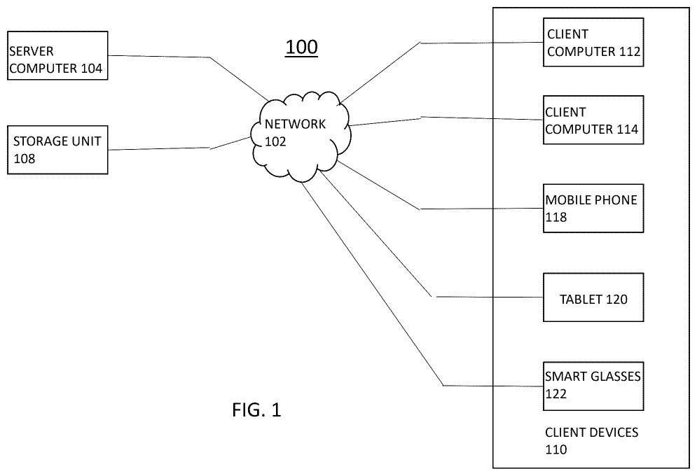

With reference to FIG. 1, a pictorial representation of a network of data processing system is depicted in which illustrative embodiments may be implemented. Network data processing system 100 is a network of computers in which the illustrative embodiments may be implemented. Network data processing system 100 contains network 102, which is the medium used to provide communications links between various devices and computers connected within network data processing system 100. Network 102 may include connections, such as wire, wireless communication links, or fiber optic cables.

In the depicted example, server computer 104 and storage unit 108 connect to network 102. In addition, client devices 110 connect to network 102. In the depicted example, server computer 104 provides information, such as boot files, operating system images, and applications to client devices 110. Client devices 110 can be, for example, computers, workstations, or network computers. As depicted, client devices 110 include client computers 112 and 114. Client devices 110 can also include other types of client devices such as mobile phone 118, tablet computer 120, and smart glasses 122.

In the illustrative example of FIG. 1, server computer, storage unit 108, and client devices 110 are network devices that connect to network 102 in which network 102 is the communications media for these network devices. Some or all of client devices 110 may form an Internet of things (IoT) in which these physical devices can connect to network 102 and exchange information with each other over network 102.

Program code located in network data processing system 100 can be stored on a computer-recordable storage medium and downloaded to a data processing system or other device for use. For example, the program code can be stored on a computer-recordable storage medium in server computer 104 and storage unit 108 and downloaded to client devices 110 over network 102 for use on client devices 110.

In the illustrative example of FIG. 1, network 102 can be the Internet representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers consisting of thousands of commercial, governmental, educational, and other computer systems that route data and messages. Of course, network data processing system 100 also may be implemented using different types of networks. For example, network 102 can be comprised an intranet, a local area network (LAN), a metropolitan area network (MAN), or a wide area network (WAN). FIG. 1 is intended as an example, and not as an architectural limitation for the different illustrative embodiments.

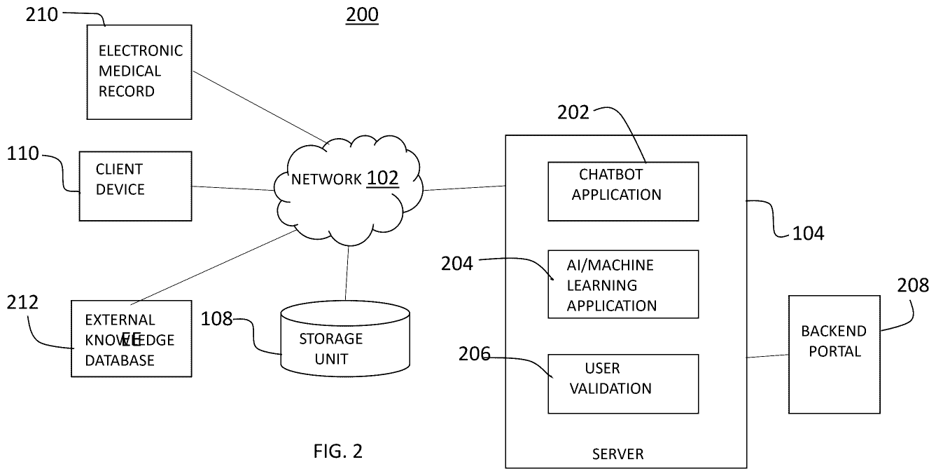

FIG. 2 provides a block diagram of system 200 in accordance with an illustrative embodiment. In one aspect, system 200 is an online platform that enables patients to receive telemedicine services and medical information remotely.

As depicted in FIG. 2, system 200 includes client device 110 which may be communicatively connected with server computer 104 via network 102. Users (e.g., patients) may access server 104 through client device 110. Client device 110 can be, for example, a network-enabled a computer or a workstation. Client device 110 can also include other types of device such as a mobile phone, a tablet computer or smart glasses. Network 102 may include the Internet. Alternatively, network 102 may include a wireless cellular network, a wide area network or any other communication network.

Server computer 104 may be equipped with or operatively coupled to a variety of HIPAA compliant remote collaboration, tele-health software programs and tools. Server computer 104 may include technologies for telemedicine and a means of providing data to and communicating with client devices 110. Server computer 104 may include technologies for secure chat and messaging, video conferencing, VOIP communication, and file sharing exchanges.

In an illustrative embodiment, server computer 104 includes chatbot application 202. Chatbot application 202 is a computer program designed to facilitate telemedicine, secure chat and messaging. Chatbot application 202 can simulate conversation with human users through text-based or voice-based interactions.

In some example embodiments chatbot application 202 implements natural language processing (NLP) to understand and generate human language. NLP enables computers to understand, interpret, and respond to human language in a way that is meaningful and contextually relevant.

Server computer 104 includes artificial intelligence (AI) application 204. AI application includes capability of a machine to imitate intelligent human behavior. It involves creating systems that can perform tasks that typically require human intelligence. These tasks include reasoning, learning, problem-solving, perception, language understanding, and interaction. AI systems are designed to perform complex functions by interpreting external data, learning from that data, and using those learnings to achieve specific goals and tasks.

In an illustrative embodiment, AI application 204 includes machine learning (ML) algorithms and statistical models that enable computers to learn and make decisions based on data. Instead of being explicitly programmed to perform a task, ML systems are trained on large amounts of data, allowing them to learn patterns and make predictions or decisions. ML algorithms within AI application 204 are configured to analyze requests and queries from users (e.g., patients) and generate appropriate responses and answers. ML algorithms are designed to understand, interpret, and respond to queries and requests in a meaningful and contextually relevant way. AI application 204 processes the inputs from users, which may include symptoms, medical history, or specific health-related questions. Based on the analysis, AI application 204 provides responses that are tailored to the user's specific situation. This can include medical advice, recommendations for further action, or answers to health-related queries. AI application 204 ensures that the responses are contextually relevant, taking into account the user's unique circumstances and health conditions. This capability enhances the user experience by providing personalized and accurate healthcare guidance. The integration of AI and machine learning in server computer 204 significantly enhances the ability to provide timely, accurate, and personalized healthcare services.

Server computer 104 includes user validation application 206 configured to authenticate and validate users (e.g., patients) that seek access to electronic health records and other confidential/protected information. A user may, for example, request information subject to HIPPA regulations. User validation application 206 may authenticate by requiring the user to provide first, last names, date of birth and social security number prior to allowing access to protected information. Additional validation procedures such as a 2-step validation using a phone number may also be required. While users can access their electronic heath records and other confidential/protected information through system 200, such information are not saved in server 104.

System 200 includes backend portal 208 which is a web-based portal configured to allow authorized service provider employees (e.g., healthcare service provider employees) to manage and control chatbot application 202. For example, backend portal 208 can be used to enable/disable features such as, for example, allow anonymous requests from users, enable/disable telehealth inquiries, enable/disable patient refill requests, and enable/disable patient lab requests. Also, backend portal 208 can be used to send request to update contact phone numbers.

In an illustrative embodiment, storage unit 108 comprises one or more hard disk drives and other types of storge devices. Storage unit 108 is configured for storing and retrieving data. Storage unit 108 may also comprise solid-state drives which use flash memory to store data. Solid state drives have no moving parts, which makes them more durable and less susceptible to physical damage. Storage unit 108 may also comprise network-attached storage (NAS) which are specialized storage devices that connect to network 102, allowing multiple users or devices to access shared storage. Storage unit 108 may also comprise cloud storage which involves storing data on remote servers accessible over the internet. Cloud storage can be used for data backup, file sharing, and remote access to files from different devices.

System 200 includes electronic health record database 210. Electronic health record database 210 is an external database or repository which retains electronic health records and other confidential/protected information subject to HIPPA regulations. After a user is authenticated and validated by user validation application 206, the user may be provided access to his/her electronic health records via chatbot application 202. For example, a user may download prescriptions, test results, scheduled visits to healthcare providers from electronic medical records 210.

System 200 includes external knowledge database 212 which is a repository of medical knowledge and training information. In response to user requests/queries which comprise general medical information, AI application 204 can retrieve relevant information from storage unit 108. If the relevant information is not found in storage unit, AI application 204 can search external knowledge database 212 and retrieve the information for the user. If the user requests information that are confidential or protected under HIPPA regulations, AI application 204 routes the requests to electronic health record 210 and retrieves the information if the user has been authenticated and validated by user validation application 206.

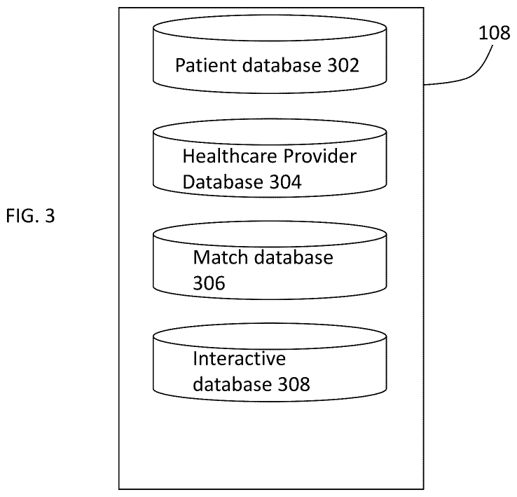

In an illustrative embodiment, storage unit 108 may include one of more databases as illustrated in FIG. 3. These databases may be HIPPA-compliant databases. Storage unit 108 may include patient database 302 configured to store information about patients who have registered with system 200. Patient database 302 is crucial for identifying and managing patient records and ensuring the privacy and security of their health information. In an illustrative embodiment, information stored in patient database 302 may include patient demographics (e.g., name, age, gender, contact information), medical history and records (diagnoses, allergies, medications), insurance information, contact preferences (email, phone, etc.) and login credentials (username and password). Patient database 302 may allow for patient registration, authentication, and secure access to system 200. It helps maintain a complete medical history for each patient, making it available to healthcare providers for accurate diagnosis and treatment.

Storage unit 108 includes healthcare provider database 304 configured to store information about healthcare professionals who offer their services through system 200. Healthcare provider database 304 is essential for ensuring that patients can connect with qualified healthcare providers. Information stored in healthcare provider database 304 may include provider credentials and qualifications (e.g., medical licenses, specialties), contact information, availability schedule (consultation hours), and billing and payment details. Healthcare provider database 304 enables patients to search for healthcare providers based on their needs, specialties, and availability. It also helps maintain a reliable directory of healthcare professionals who can provide medical services through the telemedicine platform.

Storage unit 108 includes match database 306 which is responsible for connecting patients with suitable healthcare providers based on their specific needs and preferences. In an illustrative embodiment, match database 306 connects patients with HIV healthcare service providers based on patient inquiry and zip codes. Match database 306 ensures that patients receive appropriate care from the available providers.

In an illustrative embodiment, match database 306 includes patient preferences (e.g., preferred language, gender of the provider), healthcare provider specialties, and geographical location or time zone.

In an example embodiment, match database 306 uses algorithms to analyze patient requests and provider profiles, considering factors like medical expertise, availability, geographical locations and patient preferences. It then suggests or matches patients with suitable healthcare providers.

Storage unit 108 includes interactive database 308 which serves as the platform for real-time communication and interaction between patients and healthcare providers. In an example embodiment, interactive database 308 enables chat sessions or messaging for remote consultations and medical advice. In response to a patient inquiry, interactive database 308 provides information relating to treatment centers to patients. In an example embodiment, interactive database 308 includes communication logs (e.g., chat transcripts, call records), patient and provider interactions (e.g., diagnoses, treatment plans, prescriptions), and appointment scheduling and reminders. Interactive database 308 allows patients and healthcare providers to communicate effectively and securely. In an example embodiment, interactive database 308 supports features such as video conferencing for telehealth consultations, secure messaging for discussing medical concerns, and appointment scheduling for follow-up care.

In an illustrative embodiment, databases 302, 304, 306 and 308 may include suitable types of application or data structure that may be configured as a data repository. For example, the databases may be configured as relational databases that include one or more tables of columns and rows that may be searched or queried according to a query language. Alternatively, the databases may be configured as structured data stores that include data records formatted according to a markup language, such as a version of Extensible Markup Language (XML). In other embodiments, the databases may be implemented using arbitrarily or minimally structured data files managed and accessible through any suitable type of application or the databases may be non-relational databases.

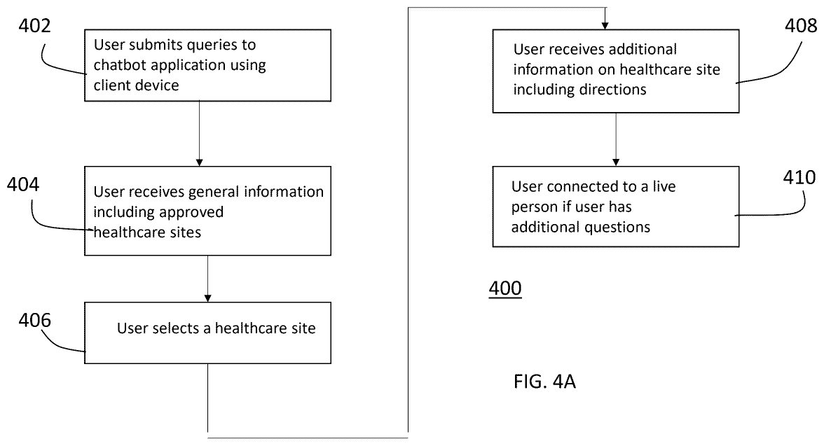

With reference next to FIG. 4A, a flowchart of process 400 for submitting user queries and requests and obtaining information is depicted in accordance with an illustrative embodiment. A user (e.g., patient) submits one or more queries on the chatbot application (step 402). The user may initiate a conversation with the chatbot application through a mobile device, a laptop computer and the like. In an example embodiment, the chatbot application may present the user with a list of predefined or preconfigured questions. The user may be prompted by the chatbot application to select one or more these predefined questions that are most relevant to the user's current health issue or reason for seeking assistance.

Once the user selects a predefined question or submits a query, the chatbot application provides a response or follow-on based on the chosen topic (step 404). In an example embodiment, the user receives a list of approved healthcare sites. The user selects a healthcare site from the list of approved sites (step 406). Once the user selects a healthcare site, the user is presented with additional information about the healthcare site including, for example, directions to the site (step 408). In some example embodiments, the user is connected to a live person (e.g., customer service representative) if the user has additional questions and desires to speak with a live person (step 410). If a live person is unavailable at that time, the chatbot application automatically sends an email to the customer service representative to contact the users/patients later.

With reference next to FIG. 4B, a flowchart of process 420 for registering with system 200 and obtaining electronic health record and other confidential information is depicted.

A user (e.g., patient) submits a request to register with system 200 (step 422). The user is then prompted to submit authentication/validation information and in response the user submits the requested information (step 424). Upon authentication and validation, the user receives registration from system 200 (step 426). The user then requests access to electronic health record or other confidential information associated with user (step 428). Next, the user receives the requested information from system 200 (step 430)

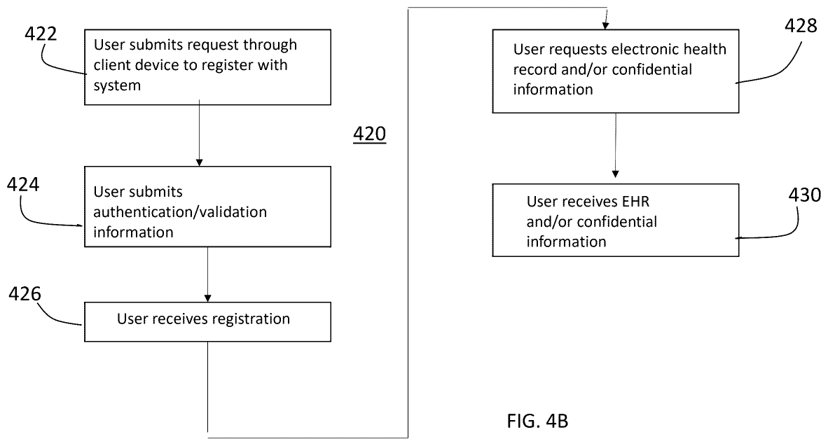

With reference to FIG. 5A, a flowchart of process 500 for providing healthcare information to a user (e.g., patient) is depicted in accordance with an illustrative embodiment. The chatbot application receives one or more queries from a user (step 502). The queries may relate to general healthcare information, the user's healthcare needs, the type of healthcare provider the user is looking for, specific medical specialties, or any other relevant details. In an example embodiment, the queries relate to HIV testing, HIV educated, clinic location, etc.

The AI application within the system processes the user's queries, extracting key information, such as the type of healthcare provider required and any specific criteria the patient may have (step 504). The AI application may also ask follow-up questions to clarify the user's needs further. For example, the AI application may ask the user for zip code or location to help narrow down the search for healthcare providers. This location data is crucial for finding healthcare sites that are convenient and accessible to the user.

Using the information provided by the user, including their queries and zip code, the AI application searches a database within the system for relevant information. If the relevant information is not found in the database within the system, the AI application searches the external knowledge database to retrieve the information (step 506). The AI application may identify a list of suitable healthcare sites based on the user's preferences and location. The AI application may consider factors like the provider's specialty, proximity to the user's location, and availability.

The AI application generates a response including a list of suitable healthcare sites and transmits this information back to the user (step 508). The user can review the response, which may include the names and contact details of the healthcare sites.

The user reviews the response including list of healthcare sites and selects one that meets the user's needs and preferences. The user communicates the selection to chatbot application (step 510).

Once the user makes a selection, the AI application responds by providing additional information about the chosen healthcare site (step 512). This information may include: address and contact details of the healthcare site; directions to the facility, including a map or written instructions; information about the healthcare provider's operating hours; and any specific requirements or preparations the user should be aware of.

This process streamlines users' search for healthcare providers, making it more convenient and efficient. It also ensures that users have access to crucial information, including directions and other details, to help them reach their chosen healthcare site with ease.

With reference to FIG. 5B, a flowchart of process 500 for user registration with the system is provided. In an example embodiment, the user is required to register with the system prior to receiving electronic health records and other confidential information subject to HIPPA regulation. The system receives a request from the user to register (step 522). The request may be received via the Chatbot application.

In response to the user request to register, the system requests authentication and validation information from the user (step 524). The user may be required to provide first and last names, date of birth, social security number and other identifying information to register. The user provides the required information which is received by the system (step 526). The system validates and authenticates the user and transmits the registration to the user (step 526). Following registration, the Chatbot application receives a request from the user to access electronic health records (step 528). The AI application with the system processes the user request and retrieves relevant information from the external database that stores electronic health records.

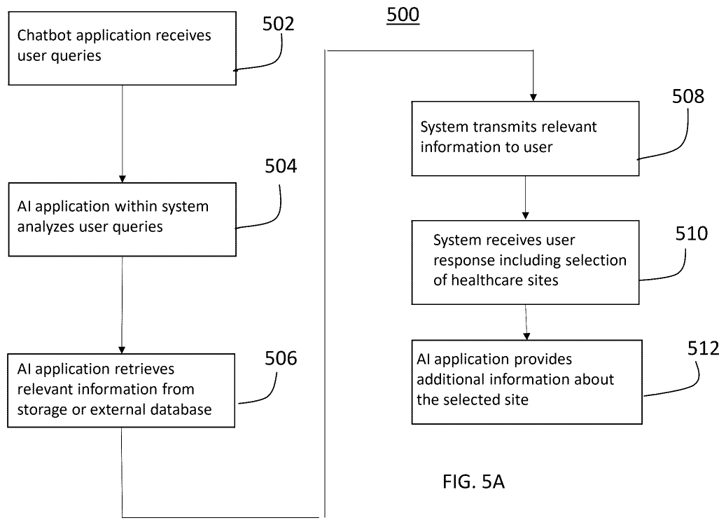

FIG. 6 shows a screen view of graphical user interface (GUI) 600 of the chatbot application in accordance with an illustrative embodiment. Features of the chatbot application can be accessed through GUI 600 on a computer screen. GUI 600 includes a welcome message to a user. As depicted in FIG. 6, GUI 600 presents a user with a list of predefined or preconfigured questions. The user is prompted to select one or more these predefined questions that are most relevant to the user's current health issue or reason for seeking assistance. Additionally, GUI 600 allows the user to send a message to the chatbot application.

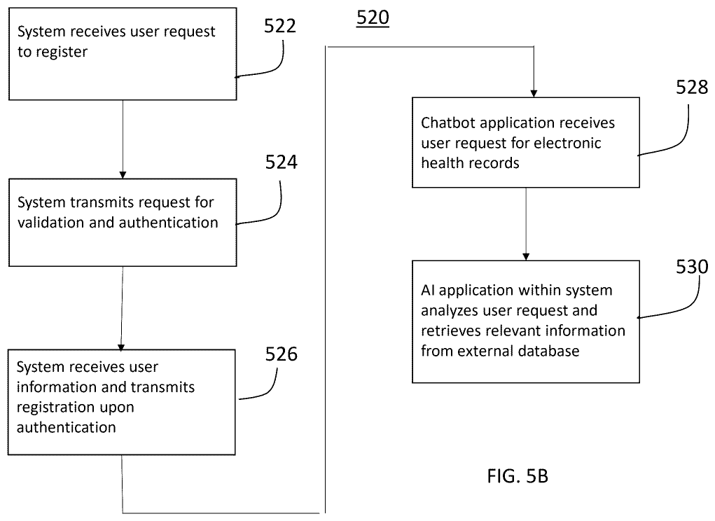

FIG. 7 shows a screen view of graphical user interface (GUI) 700 of the chatbot application in accordance with an illustrative embodiment. Features of the chatbot application can be accessed through GUI 700 on a screen. GUI 700 allows a user to receive information (e.g., HIV education, clinic location, telehealth, etc.). GUI 700 presents the user with a list of predefined or preconfigured questions. The user is prompted to select one or more these predefined questions that are most relevant to the patient's current health issue or reason for seeking assistance. Additionally, GUI 700 allows the user to send a message to the chatbot application.

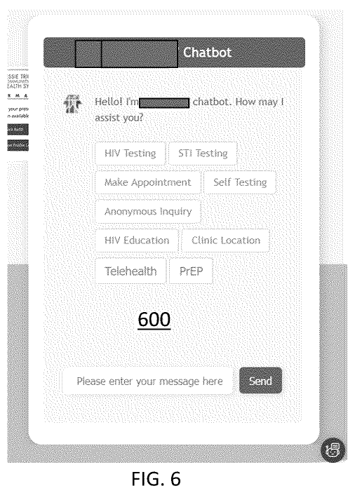

FIG. 8 shows a screen view of graphical user interface (GUI) 800 of the chatbot application in accordance with an illustrative embodiment. Once the user has selected one or more predefined questions, the chatbot application responds by providing information. The chatbot application may search a database for information. In the example of FIG. 8, the chatbot provides an explanation of telehealth to the user.

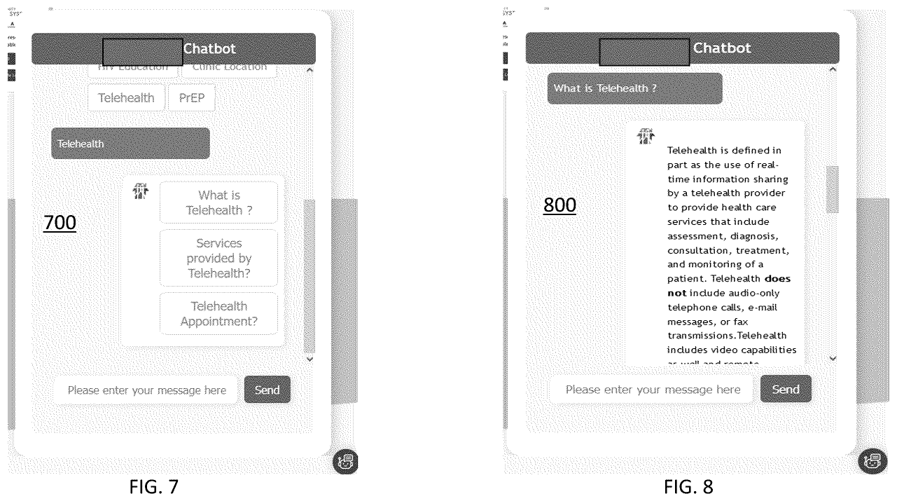

FIG. 9 shows a screen view of graphical user interface (GUI) 900 of the chatbot application in accordance with an illustrative embodiment. Features of the chatbot application can be accessed through GUI 900 on a screen. GUI 900 allows a user to make an appointment at a treatment center, virtually visit the treatment center and chat with an agent or a representative.

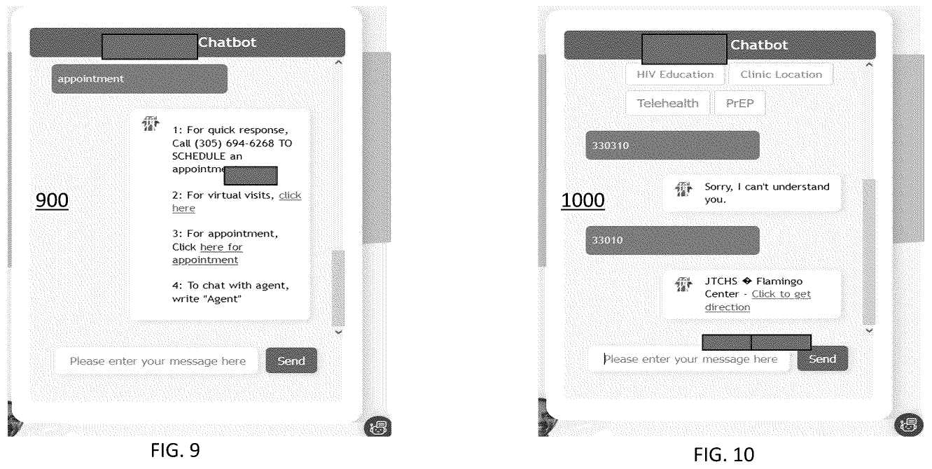

FIG. 10 shows a screen view of graphical user interface (GUI) 1000 of the chatbot application in accordance with an illustrative embodiment. Features of the chatbot application can be accessed through GUI 1000 on a screen. GUI 1000 illustrates secure chat and messaging features of the chatbot application. GUI 1000 also illustrates conversation with human users through text-based interactions. In this example, the user initially entered an invalid zip code, and the chatbot application responded “Sorry, I can't understand you.” The user thereafter entered a valid zip code and in response, the chatbot application provided the address of a healthcare site within the zip code.

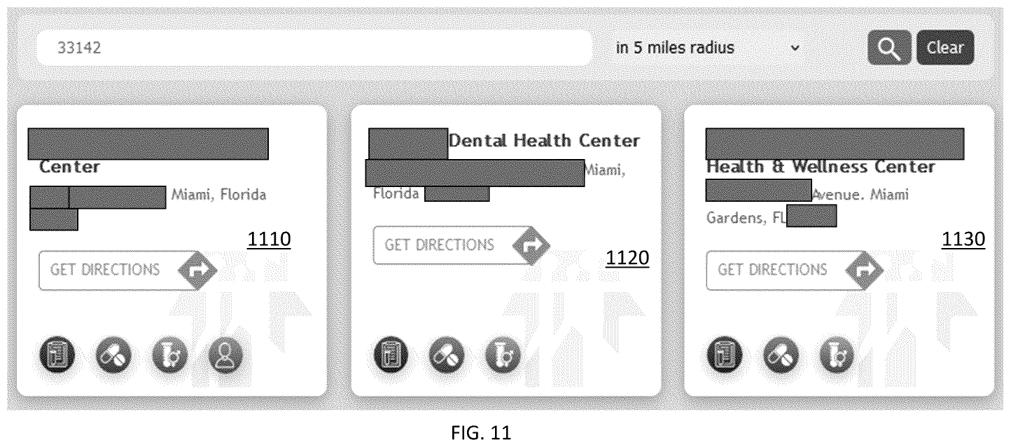

FIG. 11 illustrates graphical user interfaces (GUIs) 1110, 1120 and 1130 of the chatbot application in accordance with an illustrative embodiment. Features of the chatbot application can be accessed through GUIs 1110, 1120 and 1130 on a computer screen. GUIs 1110, 1120 and 1130 provides addresses of healthcare providers. The user can select one the healthcare providers and receive directions to the healthcare provider.

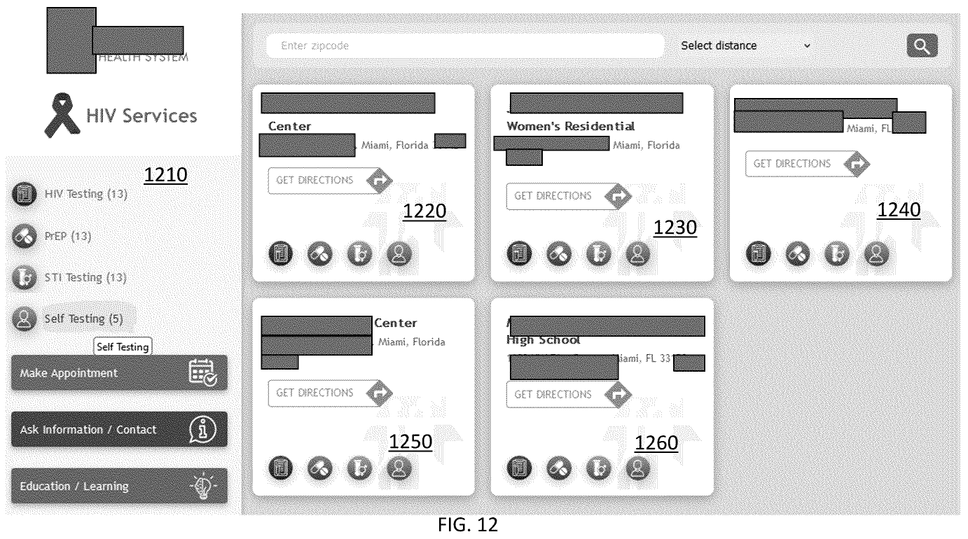

FIG. 12 shows graphical user interfaces (GUIs) 1210, 1220, 1230, 1240, 1250 and 1260 of the chatbot application in accordance with an illustrative embodiment. Features of the chatbot application can be accessed through GUIs 1210, 1220, 1230, 1240, 1250 and 1260 on a screen. GUI 1210 illustrates HIV services including HIV testing and self-testing. Also, GUI 1210 allows a user to schedule an appointment and receive information. GUIs 1220, 1230, 1240, 1250 and 1260 illustrate screenshots of addresses of healthcare providers. The user can select one the healthcare providers and receive directions to the healthcare provider.

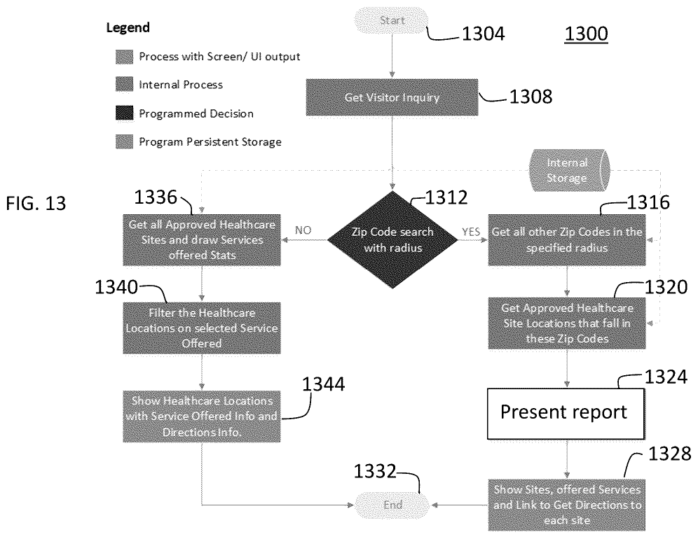

With reference to FIG. 13, a flowchart 1300 for processing user queries and providing healthcare information to a user is depicted in accordance with an illustrative embodiment. Process starts (step 1304) with the chatbot application receiving one or more user queries (step 1308). The queries relate to the user's healthcare needs such as the type of healthcare provider the user is looking for, specific medical specialties, or any other relevant details. In an example embodiment, the queries relate to general health information, medical testing, clinic location, etc.

The user may provide a search radius within which the user prefers healthcare providers to be located. The search radius helps narrow down the search for healthcare providers. If the user provides a search radius (step 1312), the chatbot application retrieves zip codes with the search radius (step 1316). The chatbot application searches a database or directory of healthcare providers. It identifies a list of suitable healthcare providers based on the search radius (step 1320).

In an example implementation, the chatbot application presents a report of medical services offered at the healthcare sites (step 1324). For example, the chatbot application may provide the user statistics relating to the types of services offered by the healthcare sites and the number of visitors at the healthcare sites.

The chatbot application compiles a list of suitable healthcare sites and transmits this information back to the user (step 1328). This information may include a link with directions to the healthcare sites. The user can review this list, which may include the names and contact details of the healthcare sites. The process then ends (step 1332).

If the user queries do not include a search radius, the chatbot searches a database or directory of healthcare providers. In an example embodiment, the chatbot application compiles a list of healthcare providers (step 1336). The chatbot can filter this list of healthcare providers (step 1340) and transmit a filtered subset of the healthcare providers to the user. The user receives the filtered list of healthcare providers (step 1344). The information received by the user may include locations and services offered by the healthcare providers and directions to the providers. The process then ends (step 1332).

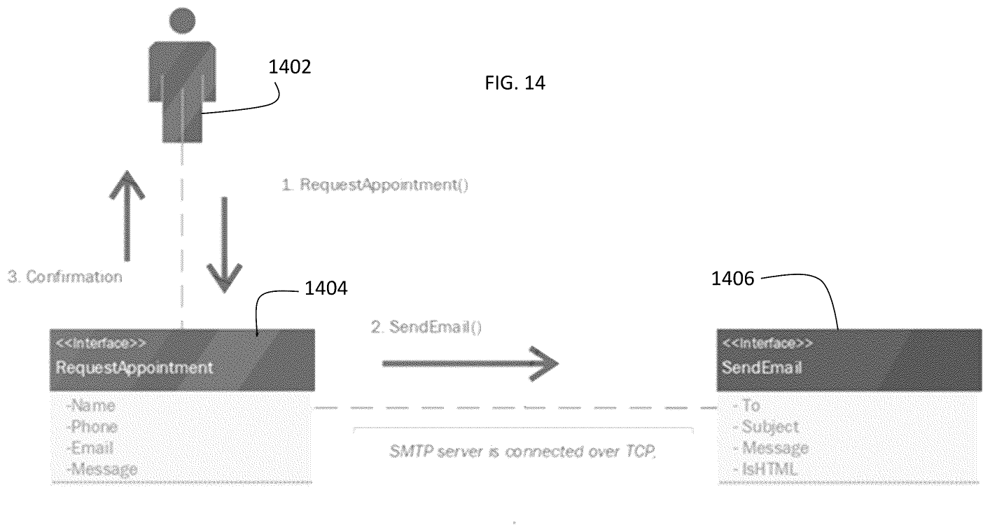

FIG. 14 is block diagram 1400 depicting messages exchanged between a user and the chatbot application in accordance with an illustrative embodiment. As depicted, user 1402 sends request 1404 to the chatbot application for an appointment. Request 1404 may be transmitted in the form of a message which may include name, phone number, email and any messages. The chatbot application may email request 1404 to email server 1406. The chatbot application may be connected to email server over a TCP connection. Email server 1406 processes the request. For example, email server 1406 may search a database or directory, identify a suitable healthcare provider and make an appointment for user 1402. The chatbot application sends a confirmation to user 1402.

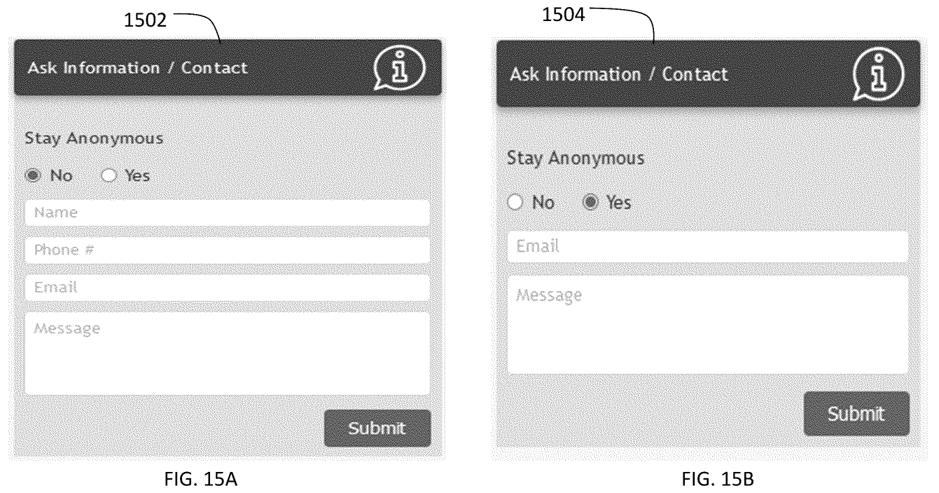

In an illustrative embodiment, the chatbot application allows a user to seek healthcare services either anonymously or as a named individual. FIG. 15A illustrates GUI 1502 through which a user may send messages as a named individual, and FIG. 15B illustrates GUI 1504 through which a user may send messages anonymously. In an example embodiment, the chatbot application sends a preconfigured confirmation message email to the user confirming that someone from a healthcare organization will be responding. Also, the chatbot application sends an email to a healthcare professional authorized to receive these questions.

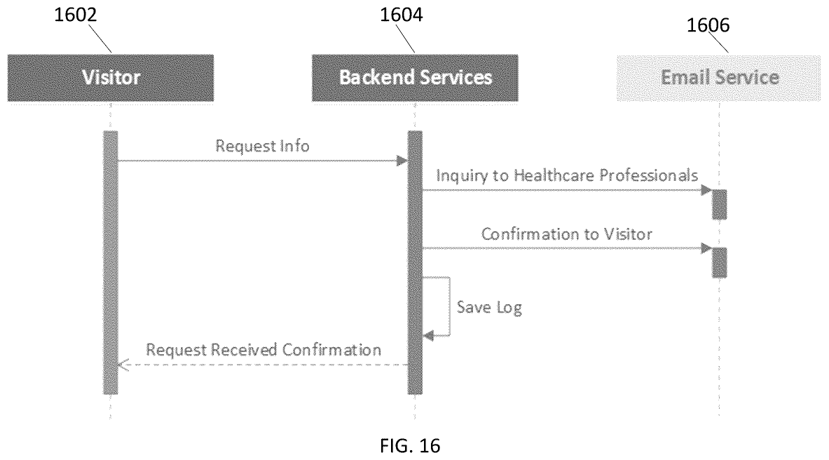

FIG. 16 illustrates a sequence of messages exchanged between user 1602, backend services 1604 and email service 1606 in accordance with an illustrative embodiment. As depicted in FIG. 16, user 1602 (visitor) sends a request for information using the chatbot application. Backend services 1604 forwards the request to email service 1606. Backend services 1604 saves the request and a confirmation in a log. Backend services 1604 respond to the request and may provide a confirmation.

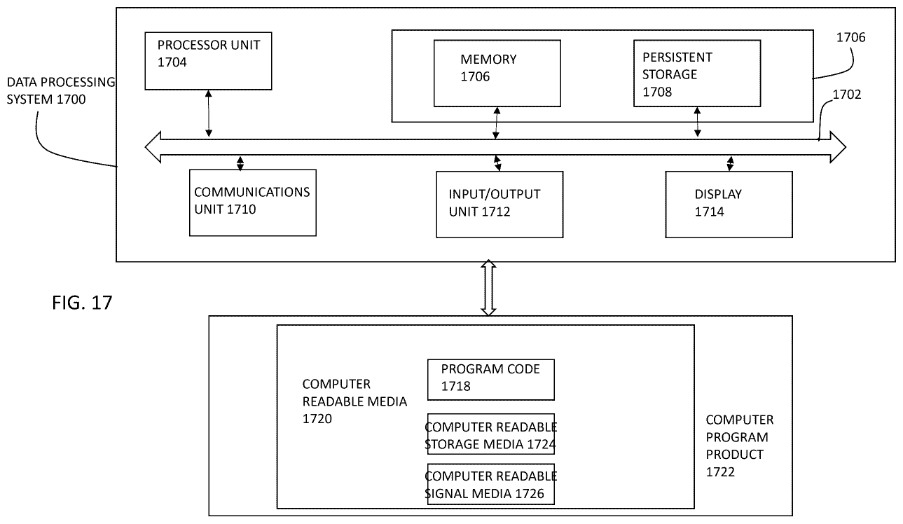

Turning now to FIG. 17, an illustration of a block diagram of a data processing system is depicted in accordance with an illustrative embodiment. Data processing system 1700 may be used to implement server computers 104 and 106 and client devices 110 in FIG. 1, as well as chatbot application 202 in FIG. 2. In this illustrative example, data processing system 1700 includes communications framework 1702, which provides communications between processor unit 1704, memory 1706, persistent storage 1708, communications unit 1710, input/output unit 1712, and display 1714. In this example, communications framework 1702 may take the form of a bus system.

Processor unit 1704 serves to execute instructions for software that may be loaded into memory 1706. Processor unit 1704 may be a number of processors, a multi-processor core, or some other type of processor, depending on the particular implementation. In an embodiment, processor unit 1704 comprises one or more conventional general-purpose central processing units (CPUs). In an alternate embodiment, processor unit 1704 comprises one or more graphical processing units (GPUs).

Memory 1706 and persistent storage 1708 are examples of storage devices 1716. A storage device is any piece of hardware that is capable of storing information, such as, for example, without limitation, at least one of data, program code in functional form, or other suitable information either on a temporary basis, a permanent basis, or both on a temporary basis and a permanent basis. Storage devices 1716 may also be referred to as computer-readable storage devices in these illustrative examples. Memory 1706, in these examples, may be, for example, a random access memory or any other suitable volatile or non-volatile storage device. Persistent storage 1708 may take various forms, depending on the particular implementation.

For example, persistent storage 1708 may contain one or more components or devices. For example, persistent storage 1708 may be a hard drive, a flash memory, a rewritable optical disk, a rewritable magnetic tape, or some combination of the above. The media used by persistent storage 1708 also may be removable. For example, a removable hard drive may be used for persistent storage 1708. Communications unit 1710, in these illustrative examples, provides for communications with other data processing systems or devices. In these illustrative examples, communications unit 1710 is a network interface card.

Input/output unit 1712 allows for input and output of data with other devices that may be connected to data processing system 1700. For example, input/output unit 1712 may provide a connection for user input through at least one of a keyboard, a mouse, or some other suitable input device. Further, input/output unit 1712 may send output to a printer. Display 1714 provides a mechanism to display information to a user.

Instructions for at least one of the operating system, applications, or programs may be located in storage devices 1716, which are in communication with processor unit 1704 through communications framework 1702. The processes of the different embodiments may be performed by processor unit 1704 using computer-implemented instructions, which may be located in a memory, such as memory 1706.

These instructions are referred to as program code, computer-usable program code, or computer-readable program code that may be read and executed by a processor in processor unit 1704. The program code in the different embodiments may be embodied on different physical or computer-readable storage media, such as memory 1706 or persistent storage 1708.

Program code 1718 is located in a functional form on computer-readable media 1720 that is selectively removable and may be loaded onto or transferred to data processing system 1700 for execution by processor unit 1704. Program code 1718 and computer-readable media 1720 form computer program product 1722 in these illustrative examples. In one example, computer-readable media 1720 may be computer-readable storage media 1724 or computer-readable signal media 1726.

In these illustrative examples, computer-readable storage media 1724 is a physical or tangible storage device used to store program code 1718 rather than a medium that propagates or transmits program code 1718. Computer readable storage media 1724, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Alternatively, program code 1718 may be transferred to data processing system 1700 using computer-readable signal media 1726. Computer-readable signal media 1326 may be, for example, a propagated data signal containing program code 1718. For example, computer-readable signal media 1726 may be at least one of an electromagnetic signal, an optical signal, or any other suitable type of signal. These signals may be transmitted over at least one of communications links, such as wireless communications links, optical fiber cable, coaxial cable, a wire, or any other suitable type of communications link.

The different components illustrated for data processing system 1300 are not meant to provide architectural limitations to the manner in which different embodiments may be implemented. The different illustrative embodiments may be implemented in a data processing system including components in addition to or in place of those illustrated for data processing system 1100. Other components shown in FIG. 17 can be varied from the illustrative examples shown. The different embodiments may be implemented using any hardware device or system capable of running program code 1718.

As used herein, “a number of,” when used with reference to items, means one or more items. For example, “a number of different types of networks” is one or more different types of networks.

Further, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, or item C” may include item A, item A and item B, or item B. This example also may include item A, item B, and item C or item B and item C. Of course, any combinations of these items can be present. In some illustrative examples, “at least one of” can be, for example, without limitation, two of item A; one of item B; and ten of item C; four of item B and seven of item C; or other suitable combinations.

The flowcharts and block diagrams in the different depicted embodiments illustrate the architecture, functionality, and operation of some possible implementations of apparatuses and methods in an illustrative embodiment. In this regard, each block in the flowcharts or block diagrams can represent at least one of a module, a segment, a function, or a portion of an operation or step. For example, one or more of the blocks can be implemented as program code, hardware, or a combination of the program code and hardware. When implemented in hardware, the hardware may, for example, take the form of integrated circuits that are manufactured or configured to perform one or more operations in the flowcharts or block diagrams. When implemented as a combination of program code and hardware, the implementation may take the form of firmware. Each block in the flowcharts or the block diagrams may be implemented using special purpose hardware systems that perform the different operations or combinations of special purpose hardware and program code run by the special purpose hardware.

In some alternative implementations of an illustrative embodiment, the function or functions noted in the blocks may occur out of the order noted in the figures. For example, in some cases, two blocks shown in succession may be performed substantially concurrently, or the blocks may sometimes be performed in the reverse order, depending upon the functionality involved. Also, other blocks may be added in addition to the illustrated blocks in a flowchart or block diagram.

The different illustrative examples describe components that perform actions or operations. In an illustrative embodiment, a component may be configured to perform the action or operation described. For example, the component may have a configuration or design for a structure that provides the component an ability to perform the action or operation that is described in the illustrative examples as being performed by the component.

Many modifications and variations will be apparent to those of ordinary skill in the art. Further, different illustrative embodiments may provide different features as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

What is claimed is:

1. A computer-implemented method for facilitating telemedicine service and information retrieval, comprising:
receiving, by a server computer from a client device over a network, a user request for telemedicine services, wherein the user request is received through a chatbot application for facilitating the telemedicine service;
analyzing the user request using an AI application on the server computer, wherein the AI application includes machine learning algorithms configured to interpret the user request;
generating a response to the user request based on the analysis;
transmitting the generated response to the client device through the chatbot application;
connecting the user to a customer service representative if the user has additional questions; and
sending an email to the customer service representative with a request to contact the user.

2. The method of claim 1, wherein the client device is a computer or a mobile phone.

3. The method of claim 1, wherein the AI application includes machine learning algorithms configured to analyze symptoms, medical history, and specific health-related questions.

4. The method of claim 1, further comprising:
receiving a request from the client device to access electronic health records;
authenticating the user using a user validation application configured to verify the user's identity; and
providing access to the user to the electronic health records upon authentication.

5. The method of claim 4, wherein the user is authenticated upon receiving first name, last name, date of birth, and social security number.

6. A computer-implemented method for facilitating telemedicine service and information retrieval, comprising:
receiving, from a user, one or more user queries related to healthcare needs through a chatbot application;
presenting predefined questions to the user for selection based on the user's current health issues or reason for seeking assistance;
providing a response based on the selected predefined question;
offering a list of approved healthcare sites to the user;
receiving the user's selection of a healthcare site from the offered list;
presenting additional information about the selected healthcare site, including directions to the site;
connecting the user to a customer service representative if the user has additional questions; and
sending an email to the customer service representative with a request to contact the user.

7. The computer-implemented method of claim 6, wherein the user's selection determines the subsequent information provided by the chatbot application.

8. The computer-implemented method of claim 6, wherein an AI application processes the user's queries to extract information including the type of healthcare provider required and location-related criteria to refine the search for suitable healthcare sites.

9. The computer-implemented method of claim 6, wherein the additional information provided about the selected healthcare site includes address, contact information, operating hours, and specific user preparations.

10. The computer-implemented method of claim 6, wherein the chatbot application is configured to process natural language queries.

11. A system for facilitating telemedicine service, comprising:
a storage device configured to store program instructions; and
one or more processors operably connected to a communication network and the storage device and configured to execute the program instructions to cause the system to:
receive, from a client device over the communication network, a user request for telemedicine services, wherein the user request is received through a chatbot application for facilitating the telemedicine service;
analyze the user request using an AI application, wherein the AI application includes machine learning algorithms configured to interpret the user request;
generate a response to the user request based on the analysis;
transmit the generated response to the client device using the chatbot application;
connect the user to a customer service representative if the user has additional questions; and
send an email to the customer service representative with a request to contact the user.

12. The system of claim 11, wherein the program instructions cause the system to:
receive a request from the client device to access electronic health records;
authenticate the user using a user validation application configured to verify the user's identity; and
provide access to the user to the electronic health records upon authentication.

13. The system of claim 11, wherein the program instructions cause the system to:
receive, from the client device, one or more user queries related to healthcare needs through the chatbot application;
present predefined questions to the client device for selection based on the user's current health issues or reason for seeking assistance;
provide a response based on the selected predefined question; and
offer a list of approved healthcare sites to the user.

14. The system of claim 13, wherein the program instructions cause the system to:
receive the user's selection of a healthcare site from the list; and
present additional information about the selected healthcare site, including directions to the site.

15. The system of claim 11, wherein the AI application processes the user's queries to extract information including the type of healthcare provider required and location-related criteria to refine the search for suitable healthcare sites.

16. The system of claim 11, wherein the client device is a computer or a mobile phone.

17. The system of claim 11, wherein the storage unit comprises a match database configured to connect the user with one or more suitable healthcare sites.

18. The system of claim 11, wherein the chatbot application is a secure messaging application configured to facilitate exchange of messages between the user and the chatbot application.

19. The system of claim 11, wherein the chatbot application offers follow-up questions to clarify the user's healthcare needs, including location data, to facilitate narrowing down the search for healthcare providers.

20. The system of claim 11, wherein the chatbot application compiles and transmits the list of suitable healthcare sites to the user, including names and contact details of the healthcare sites.
